(19) United States
(12) Patent Application Publication
(10) Pub. No.: US 2023/0080653 A1
(43) Pub. Date: Mar. 16, 2023

(54) MEAT SUBSTITUTE

(71) Applicant: Marlow Foods Limited, Stokkesley (GB)

(72) Inventors: Timothy Finnigan, Hutton Rudby (GB); Muyiwa Akintoye, Guisborough (GB); Reza Mousavi, Middlesbrough (GB)

(21) Appl. No.: 17/990,954

(22) Filed: Nov. 21, 2022

Related U.S. Application Data
(63) Continuation of application No. 15/546,716, filed on Jul. 27, 2017.

(30) Foreign Application Priority Data
Jan. 27, 2015 (GB) 1501320.4

(51) Int. Cl. A23L 31/00; A23J 3/22; A23L 29/00; A23L 33/185; A23L 13/40; A23L 33/175; A23L 13/00; A23J 1/00

(52) U.S. Cl. CPC A23L 31/00; A23J 3/227; A23L 29/015; A23L 33/185; A23L 13/426; A23L 33/175; A23L 13/424; A23L 13/00; A23J 1/00; A23V 2002/00

(57) ABSTRACT

Described herein is an edible formulation, suitable for vegans, that comprises edible fungal particles of a filamentous fungus and calcium ions.

### CROSS-REFERENCE TO RELATED APPLICATIONS

This application is a continuation-in-part of U.S. patent application Ser. No. 15/546,716 filed on Jul. 27, 2017, which is a national stage entry of International Patent Application No. PCT/GB2016/050144, filed on Jan. 22, 2016, which claims priority to Great Britain Patent Application No. 1501320.4, filed on Jan. 27, 2015, the contents of each which are incorporated by reference in their entirety.

### TECHNICAL FIELD

Described herein are compositions of edible fungi and particularly, although not exclusively, edible formulations comprising edible fungal particles for use as meat substitutes.

### BACKGROUND

It is known, for example from WO 2000/15045 A1 (DSM), WO 1996/21362 A1 (Zeneca) and WO 1995/23843 A1 (Zeneca) to use edible filamentous fungi as meat-substitutes, for example in the preparation of burgers and sausages. In such uses, filaments of the fungi are bound together, for example with egg albumin, and are texturized so that the product resembles muscle fibres and therefore has a meat-like appearance and texture. Meat substitutes of the type described have been widely commercially available for many years under the trademark QUORN™.

In some circumstances, it is desirable to reduce or even eliminate the amount of egg albumin used with edible fungus in the manufacture of meat-substitutes for example on cost grounds or to produce a product suitable for vegans.

It is an object of the present invention to address the aforementioned problem.

### SUMMARY

One embodiment described herein is a meat substitute edible formulation comprising: at least 20 wt % on a dry mass basis of edible filamentous fungus particles comprising filaments of fungal mycelia; at least 0.100 wt % and less than 1 wt % on a dry mass basis of calcium ions; at least 50 wt % of water; and acetate moieties; wherein the formulation contains no animal derived components. In one aspect, the filaments of fungal mycelia have an aspect ratio of greater than 10 and a length of greater than 100 μm. In another aspect, the formulation comprises at least 2,000 mg of calcium ions per kg of filamentous fungus particles on a dry mass basis. In another aspect, the formulation comprises at least 5,000 mg of calcium ions per kg of filamentous fungus particles on a dry mass basis. In another aspect, the formulation comprises a ratio of the wt % on a dry mass basis of acetate ions divided by the wt % on a dry mass basis of filamentous fungus particles of at least 0.005. In another aspect, the formulation comprises at least 0.10 wt % on a dry mass basis and less than 1.5 wt % on a dry mass basis of acetate ions. In another aspect, the filamentous fungal particles have an RNA content of less than 1.9 wt % on a dry mass basis. In another aspect, the formulation comprises a ratio of the wt % on a dry mass basis of filamentous fungus particles divided by the wt % of water of at least 0.05 and less than 0.5. In another aspect, the formulation is provided in a package comprising at least 50 g of the formulation. In another aspect, at least 80 wt % of the filamentous fungal particles in the formulation comprise filaments of fungal mycelia. In another aspect, the filamentous fungal particles comprise fungi imperfecti. In another aspect, the filaments of fungal mycelia have an aspect ratio of greater than 10 and a length of greater than 100 μm; the formulation comprises at least 2,000 mg of calcium ions per kg of filamentous fungus particles on a dry mass basis; the formulation comprises a ratio of the wt % on a dry mass basis of acetate ions divided by the wt % on a dry mass basis of filamentous fungus particles of at least 0.005; the formulation comprises at least 0.10 wt % on a dry mass basis and less than 1.5 wt % on a dry mass basis of acetate ions; the filamentous fungal particles have an RNA content of less than 1.9 wt % on a dry mass basis; and the formulation is provided in a package comprising at least 50 g of the formulation. In another aspect, at least 80 wt % of the filamentous fungal particles in the formulation comprise filaments of fungal mycelia; the filamentous fungal particles comprise cells of a Fusarium species; and the formulation is provided in a package comprising at least 100 g of the formulation.

Another embodiment described herein is a meat substitute edible formulation comprising: at least 20 wt % on a dry mass basis of edible filamentous fungus particles; calcium ions; and acetate moieties; wherein: at least 80 wt % of the filamentous fungus particles comprise fungal mycelia; the formulation comprises at least 2,000 mg of calcium ions per kg of filamentous fungus particles on a dry mass basis; and a ratio of the wt % on a dry mass basis of acetate ions divided by the wt % on a dry mass basis of filamentous fungus particles is at least 0.005 and is less than 0.04. In another aspect, at least 80 wt % of the filamentous fungus particles comprise fungal mycelia, and the filamentous fungus particles are fungi imperfecti. In another aspect, the formulation is provided in a package comprising at least 100 g of the formulation. In another aspect, the filamentous fungus particles comprise cells of a Fusarium species, and the formulation is provided in a package comprising at least 50 g of the formulation. In another aspect, the formulation comprises at least 40 wt % on a dry mass basis of filamentous fungus particles. In another aspect, the formulation comprises at least 0.100 wt % and less than 1 wt % on a dry mass basis of calcium ions, and at least 50 wt % of water.

Another embodiment described herein is a meat substitute edible formulation comprising: at least 20 wt % on a dry mass basis of edible filamentous fungus particles, wherein at least 99 wt % of the filamentous fungus particles comprise filaments of fungal mycelia having an aspect ratio of greater than 40 and a number average length of greater than 100 μm; at least 6,000 mg of calcium ions per kg of filamentous fungus particles on a dry mass basis; and at least 50 wt % of water; wherein the formulation contains no animal derived components.

According to a first object aspect of the invention, there is provided an edible formulation comprising edible fungal particles of a filamentous fungus and calcium ions.

One class of people may find it acceptable, generally, to avoid all animal products except dairy products. Thus, such a class of people may find an edible formulation which incorporates whey protein (e.g., up to about 10 wt % whey protein) as acceptable. Thus, in one embodiment, the edible formulation includes no animal products except, optionally, dairy products. However, in a second embodiment, the edible formulation is suitable for vegans. Consequently, in this embodiment, the edible formulation preferably includes 0 wt % of components derived from animals.

Thus, it follows from the aforementioned that the formulation preferably includes 0 wt % of a hydrocolloid derived from an animal source. The formulation preferably includes 0 wt % of egg albumin.

The edible formulation is preferably a meat substitute.

The edible formulation suitably includes at least 5 wt %, preferably at least 15 wt %, more preferably at least 20 wt % of the filamentous fungus on a dry mass basis. In some cases, for example, when the edible formulation mimics a meat product (e.g., strips of meat or mince) which may be used as a food ingredient, the edible formulation may include higher levels of the filamentous fungus, for example greater than 40 wt % or greater than 60 wt % of filamentous fungus on a dry mass basis. The edible formulation may include 5 to 80 wt %, preferably 15 to 75 wt % of filamentous fungus on a dry mass basis.

The edible formulation suitably includes at least 2,000 mg, preferably at least 4,000 mg, more preferably at least 6,000 mg, especially at least 8,000 mg of calcium ions per kg of filamentous fungus on a dry mass basis. The edible formulation suitably includes less than 25,000 mg, preferably less than 20,000 mg, of the calcium ions per kg of filamentous fungus on a dry mass basis.

The edible formulation may include intracellular calcium ions (e.g., within the edible fungal particles) and extracellular calcium ions. The edible formulation suitably includes the following extracellular levels of calcium ions: at least 2,000 mg, preferably at least 4,000 mg, more preferably at least 6,000 mg, especially at least 8,000 mg per kg of filamentous fungus on a dry mass basis. The maximum extracellular level of calcium ions is suitably less than 25,000 mg, preferably less than 20,000 mg, per kg of filamentous fungus on a dry mass basis.

The total amount of calcium ions in the edible formulation is suitably at least 5,000 mg per kg, preferably at least 10,000 mg per kg, of filamentous fungus on a dry mass basis. The total amount of calcium ions may be less than 40,000 mg or less than 20,000 mg per kg of filamentous fungus.

The edible formulation may include at least 0.100 wt %, preferably at least 0.200 wt %, more preferably at least 0.300 wt %, of calcium ions in total, on a dry mass basis. It may include less than 1 wt % or less than 0.8 wt % of calcium ions in total, on a dry mass basis.

The edible formulation may include at least 50 wt %, preferably at least 60 wt %, water. The formulation may include less than 85 wt % or less than 80 wt % water. The edible formulation may include the aforementioned levels of water after a precursor of the edible formulation has been treated (e.g., cooked such as by steaming, chilling and/or freezing) to develop texture.

The edible formulation may include a polysaccharide, for example a sulphonated polysaccharide, for example carrageenan. The edible formulation, on a dry mass basis, suitably includes at least 0.1 wt %, preferably at least 0.5 wt %, more preferably at least 0.9 wt % of the polysaccharide, for example carrageenan. It may include less than 2 wt % of the polysaccharide.

The edible formulation may include an alginate, for example a salt of alginic acid such as sodium alginate. The edible formulation, on a dry mass basis, suitably includes 0.01 wt %, preferably at least 0.05 wt %, more preferably at least 0.12 wt % of alginate. It may include less than 1 wt %, preferably less than 0.5 wt % of alginate.

The edible formulation may include gluten, for example wheat gluten. The edible formulation, on a dry mass basis, suitably includes at least 0.1 wt %, preferably at least 1 wt %, more preferably at least 1.5 wt % of the gluten. It may include less than 5 wt % of the gluten.

The edible formulation may include a protein source in addition to the edible fungal particles and which is not a wheat-based protein. The protein source (A) may be a source of vegetable protein. The protein source (A) may be a potato protein source. The edible formulation, on a dry mass basis, may include at least 1 wt %, preferably at least 5 wt % of protein source (A). It may include less than 20 wt % of protein source (A).

It is found that, if calcium chloride alone is used in the formulation, even at a minimum level to achieve the effect described herein, a disagreeable taste may result. Accordingly, steps may be taken to counter the disagreeable taste. It has been found that use of calcium acetate is able to do this. Thus, the edible formulation preferably includes acetate moieties (which may have been initially incorporated into the formulation as calcium acetate).

In the edible formulation, the ratio of the wt % of acetate ions divided by the wt % of filamentous fungus on a dry mass basis is suitably at least 0.005, preferably at least 0.01. The ratio may be less than 0.04, for example less than 0.03.

The acetate ions are suitably extra-cellular ions.

The edible formulation may include at least 0.10 wt %, preferably at least 0.40 wt % of acetate ions on the dry mass basis. It may include less than 2.00 wt %, for example less than 1.00 wt % of acetate ions on a dry mass basis.

In a first preferred embodiment, the edible formulation may include:
at least 2,000 mg, for example at least 5,000 mg, of calcium ions per kg of filamentous fungus on a dry mass basis;
at least 50 wt % of water.

In a second preferred embodiment, the edible formulation may include:
5,000 mg of calcium ions per kg of filamentous fungus on a dry mass basis;
less than 25,000 mg of calcium ions per kg of filamentous fungus on a dry mass basis;
at least 50 wt % of water;
0 wt % of egg albumin.

In the first and second preferred embodiments, preferably in the edible formulation, the ratio of the wt % of acetate ions divided by the wt % of filamentous fungus on a dry mass basis is at least 0.005 and is less than 0.03.

The edible formulation preferably includes at least 0.10 wt %, preferably at least 0.50 wt %; and suitably includes less than 1.5 wt %, or less than 1.1 wt % of acetate ions, on a dry mass basis.

### DESCRIPTION OF THE DRAWINGS

Specific embodiments of the invention will now be described, by way of example, with reference to the accompanying drawings:

FIG. 1 shows a schematic diagram showing a process for producing mycoprotein paste with reduced RNA levels by direct steam injection.

FIG. 2 shows the elastic moduli of the compositions of Examples 1-3.

FIG. 3 shows a schematic representation of steps in the production of mycoprotein-containing products for human consumption.

### DETAILED DESCRIPTION

Unless otherwise defined, all technical and scientific terms used herein have the same meaning as commonly understood by one of ordinary skill in the art. For example, any nomenclatures used in connection with, and techniques of food science, chemistry, biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “by mass” (or “by weight”) and mass percent (wt % or weight percent) refer to the mass of a particular component as a percentage of the total mass of the composition.

As used herein, the term “wet mass” refers to the sum of the dry ingredient mass plus the total mass of water in an individual component. The phrase “wet mass basis” refers to the ratio or percentage of a particular component or group of components based on the wet mass. Similarly, the term “wet mass percentage” refers to the wet mass of a particular component as a percentage of the total wet mass of the composition. In one aspect, phrases such as “at least 50 wt % of water” includes the water that may be contained in an individual component.

As used herein, the term “dry mass” refers to the dry ingredient mass in the absence of any water. The phrase “dry mass basis” refers to the ratio or percentage of a particular component or group of components based on the dry mass. Similarly, the term “dry mass percentage” refers to the dry mass of a particular component as a percentage of the total dry mass of the composition.

As used herein, the term “mycoprotein paste” refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American Type Culture Collection, 12301 Parklawn Drive, Rockville Md. 20852) and treated to reduce its RNA content to less than 2% by mass by heat treatment. Further details on the material are provided in WO 1996/21362 A1 and WO 1995/23843 A1, which are incorporated by reference herein for their teachings. The material may be obtained from Marlow Foods Limited of Stokesley, U.K. It comprises about 23-25 wt % solids (the balance being water) made up of non-viable RNA reduced fungal hyphae of approximately 400-750 μm length, 3-5 μm in diameter and a branching frequency of 2-3 tips per hyphal length.

As used herein, the phrase “calcium chloride solution” refers to a 36 wt % aqueous solution of calcium chloride.

As used herein, the phrase “calcium acetate” refers to calcium acetate in solid form.

As used herein, the phrase “sodium alginate” refers to sodium alginate in a solid form.

As described, fungal particles suitably comprise a filamentous fungus. The filamentous fungus preferably comprises fungal mycelia and suitably at least 80 wt %, preferably at least 90 wt %, more preferably at least 95 wt % and, especially, at least 99 wt % of the fungal particles in the formulation comprise fungal mycelia. Some filamentous fungi may include both fungal mycelia and fruiting bodies. The fungal particles preferably comprise a filamentous fungus of a type which does not produce fruiting bodies. Where, however, a filamentous fungus of a type which produces fruiting bodies is used, the fungal particles in the composition suitably include at least 80 wt %, preferably at least 90 wt %, more preferably at least 95 wt % of fungal mycelia. Preferably, the fungal particles comprise substantially only fungal mycelia—that is, the fungal particles in the composition preferably do not include any fruiting bodies.

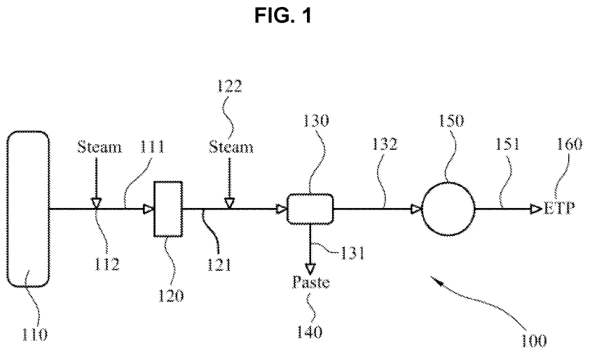

In one embodiment described herein, referring to FIG. 1, a commercially-used process (100) for producing a mycoprotein paste involves growing a fungal culture in a pressure cycle fermenter (110) at 27° C. in the presence of a growth medium. The culture broth passes from the fermenter (110) through a conduit (111) into an RNA reduction vessel (120). Steam (at 7 bar and 160° C.) is injected into the culture broth via a steam injection port (112) in the conduit (111). Steam injection raises the temperature of the culture broth to 60-70° C. Steam injection is performed to reduce the RNA content of the final mycoprotein paste (140).

The RNA reduction vessel (120) is a continuously stirred tank reactor. The culture broth is held in the RNA reduction vessel (120) at the RNA reduction temperature for at least 30 minutes. The culture broth then passes from the RNA reduction vessel (120) to centrifuges (130) via a conduit (121). Steam is injected into the culture broth via a steam injection port (122) in the conduit (121). This injection of steam increases the temperature of the culture broth to 80-90° C. for hygienic purposes. The centrifuges (130) are run at 5000×g for a period of time. The centrifuges (130) separate the mycoprotein paste (140) and waste liquid centrate. The mycoprotein paste leaves the centrifuges (130) via a conduit (131). The waste liquid centrate contains RNA and digestion products of RNA that have passed out of the fungal cells into the surrounding aqueous media. The waste liquid centrate, which at this stage has a temperature of 80-90° C., passes through conduit (132) to a cooler (150) in which it is cooled to 30° C. It then travels through conduit (151) to an effluent treatment plant (ETP) (160) for disposal. The final mycoprotein paste (140) has a nucleic acid content of less than 2% on a dry mass basis.

Preferred fungi for the fungal particles have a cell wall which includes chitin and/or chitosan. Preferred fungi have a cell wall which includes polymeric glucosamine. Preferred fungi have a cell wall which includes β1-3 and 1-6 glucans.

The fungal particles may include fungal cells of the order Mucorales as described in WO 2000/15045 A1 (DSM), which is incorporated by reference herein for such teachings.

Fungal particles preferably comprise fungus selected from fungi imperfecti.

Preferably, fungal particles comprise, and preferably consist essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, Va.) as described for example in WO 1996/21361 A1 (Zeneca) and WO 1995/23843 A1 (Zeneca), which are incorporated by reference herein for such teachings.

Preferably, the fungal particles are non-viable. Preferably, the fungal particles have been treated to lower the level of RNA which they contain. Thus, the level of RNA in the fungal particles used is preferably less than the level in an identical fungus when in a viable state. The level of RNA may be reduced as described in WO 1995/23843 A1, which is incorporated by reference herein for such teachings. The fungal particles suitably have an RNA content on a dry mass basis of less than 1.9 wt %, for example 1.7 wt % or less.

Fungal particles in the formulation may comprise filaments having lengths of less than 1000 μm, preferably less than 800 μm. The filaments may have a length greater than 50 μm, preferably greater than 100 μm, more preferably greater than 200 μm. Preferably, fewer than 5 wt %, preferably substantially no, fungal particles in the formulation have lengths of greater than 5000 μm; and preferably fewer than 5 wt %, preferably substantially no, fungal particles have lengths of greater than 2500 μm. Preferably, values for the number average of the lengths of the fungal particles in the formulation are also as stated above.

Fungal particles in the formulation may comprise filaments having diameters of less than 20 μm, preferably less than 10 μm, more preferably 5 μm or less. The filaments may have diameters greater than 1 μm, preferably greater than 2 μm. Preferably, values for the number average of the diameters of the fungal particles in the formulation are also as stated above.

Fungal particles in the formulation may comprise filaments having an aspect ratio (length/diameter) of less than 1000, preferably less than 750, more preferably less than 500, especially of 250 or less. The aspect ratio may be greater than 10, preferably greater than 40, more preferably greater than 70. Preferably, values for the average aspect ratio of the fungal particles (i.e., the average of the lengths of the particles divided by the average of the diameters of the fungal particles) in the formulation are also as stated above.

In the edible formulation, the ratio of the wt % of filamentous fungus (on a dry mass basis) divided by the wt % of water is at least 0.05, preferably at least 0.10. The ratio may be less than 0.5 or less than 0.4.

The edible formulation may be provided in a package. Thus, the edible formulation is suitably surrounded by packaging material which may comprise a receptacle. The package may include at least 50 g or at least 100 g of the edible formulation. The package may include the edible formulation and include at least 10 g of edible fungal particles on a dry mass basis. The package may include an edible formulation which includes at least 20 wt %, for example at least 45 wt % water.

According to a second aspect of the invention, there is provided a method of making an edible formulation, the method comprising:

(i) selecting a formulation comprising edible fungal particles of a filamentous fungus;

(ii) contacting the formulation with calcium ions.

Contact may be affected so as to produce an edible formulation with calcium ions as described in the first aspect and/or at levels described in the first aspect. Thus, the edible formulation prepared may be as described in the first aspect.

The edible fungal particles selected in step (i) are suitably fungal particles which have been treated after removal from a reactor in which the particles are produced in a fermentation process.

The edible fungal particles selected in step (i) are preferably not viable.

The edible fungal particles selected in step (i) preferably have been treated to lower the level of RNA they contain.

The edible fungal particles selected in step (i) preferably have an RNA content on a dry mass basis of less than 1.9 wt %, for example 1.7 wt % or less.

Any feature of any aspect of any invention described herein may be combined with any other invention described herein mutatis mutandis.

One embodiment described herein is a meat substitute edible formulation comprising: at least 20 wt % on a dry mass basis of edible filamentous fungus particles comprising filaments of fungal mycelia; at least 0.100 wt % and less than 1 wt % on a dry mass basis of calcium ions; at least 50 wt % of water; and acetate moieties; wherein the formulation contains no animal derived components. In one aspect, the filaments of fungal mycelia have an aspect ratio of greater than 10 and a length of greater than 100 μm. In another aspect, the formulation comprises at least 2,000 mg of calcium ions per kg of filamentous fungus particles on a dry mass basis. In another aspect, the formulation comprises at least 5,000 mg of calcium ions per kg of filamentous fungus particles on a dry mass basis. In another aspect, the formulation comprises a ratio of the wt % on a dry mass basis of acetate ions divided by the wt % on a dry mass basis of filamentous fungus particles of at least 0.005. In another aspect, the formulation comprises at least 0.10 wt % on a dry mass basis and less than 1.5 wt % on a dry mass basis of acetate ions. In another aspect, the filamentous fungal particles have an RNA content of less than 1.9 wt % on a dry mass basis. In another aspect, the formulation comprises a ratio of the wt % on a dry mass basis of filamentous fungus particles divided by the wt % of water of at least 0.05 and less than 0.5. In another aspect, the formulation is provided in a package comprising at least 50 g of the formulation. In another aspect, at least 80 wt % of the filamentous fungal particles in the formulation comprise filaments of fungal mycelia. In another aspect, the filamentous fungal particles comprise fungi imperfecti. In another aspect, the filaments of fungal mycelia have an aspect ratio of greater than 10 and a length of greater than 100 μm; the formulation comprises at least 2,000 mg of calcium ions per kg of filamentous fungus particles on a dry mass basis; the formulation comprises a ratio of the wt % on a dry mass basis of acetate ions divided by the wt % on a dry mass basis of filamentous fungus particles of at least 0.005; the formulation comprises at least 0.10 wt % on a dry mass basis and less than 1.5 wt % on a dry mass basis of acetate ions; the filamentous fungal particles have an RNA content of less than 1.9 wt % on a dry mass basis; and the formulation is provided in a package comprising at least 50 g of the formulation. In another aspect, at least 80 wt % of the filamentous fungal particles in the formulation comprise filaments of fungal mycelia; the filamentous fungal particles comprise cells of a Fusarium species; and the formulation is provided in a package comprising at least 100 g of the formulation.

Another embodiment described herein is a meat substitute edible formulation comprising: at least 20 wt % on a dry mass basis of edible filamentous fungus particles; calcium ions; and acetate moieties; wherein: at least 80 wt % of the filamentous fungus particles comprise fungal mycelia; the formulation comprises at least 2,000 mg of calcium ions per kg of filamentous fungus particles on a dry mass basis; and a ratio of the wt % on a dry mass basis of acetate ions divided by the wt % on a dry mass basis of filamentous fungus particles is at least 0.005 and is less than 0.04. In another aspect, at least 80 wt % of the filamentous fungus particles comprise fungal mycelia, and the filamentous fungus particles are fungi imperfecti. In another aspect, the formulation is provided in a package comprising at least 100 g of the formulation. In another aspect, the filamentous fungus particles comprise cells of a Fusarium species, and the formulation is provided in a package comprising at least 50 g of the formulation. In another aspect, the formulation comprises at least 40 wt % on a dry mass basis of filamentous fungus particles. In another aspect, the formulation comprises at least 0.100 wt % and less than 1 wt % on a dry mass basis of calcium ions, and at least 50 wt % of water.

Another embodiment described herein is a meat substitute edible formulation comprising: at least 20 wt % on a dry mass basis of edible filamentous fungus particles, wherein at least 99 wt % of the filamentous fungus particles comprise filaments of fungal mycelia having an aspect ratio of greater than 40 and a number average length of greater than 100 μm; at least 6,000 mg of calcium ions per kg of filamentous fungus particles on a dry mass basis; and at least 50 wt % of water; wherein the formulation contains no animal derived components.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

In the examples that follow, Examples 1-4 show the preparation and testing of samples which include calcium or sodium ions to illustrate the advantageous effect produced when calcium ions are used. Example 5 provides a general method of producing mycoprotein-containing products. Examples 6-9 provide details for the manufacturing specific products which are suitable for consumption by vegans. It was found that the addition of calcium cations (via calcium chloride and/or calcium acetate) to the ingredients described in the examples produces a rise in firmness and produces acceptable quality of the product produced. Without being bound by any theory, it is thought that the calcium cations interact with the mycoprotein paste to increase its firmness and strength.

### Examples 1-3—Sample Preparation

Formulations were prepared by mixing mycoprotein with either calcium chloride solution (Example 2) or sodium chloride solution (Example 3) to introduce calcium ions or sodium ions (at the same concentration) into the mycoprotein. Example 1 was a control which consisted of mycoprotein with no added sodium or calcium salts. In each case, the formulations were formed into rectangular pieces having 15 mm thickness using a forming machine.

Samples were treated differently as follows:
Treatment 1 (T1)—Samples stored under ambient conditions.
Treatment 2 (T2)—Samples steamed at 100° C. for 24 minutes.
Treatment 3 (T3)—Samples subjected to Treatment 2, followed by freezing at −21° C. for at least one week.

### Example 4—Sample Testing

To analyse rheology, the elastic modulus (G′ (Pa)) of mycoprotein containing formulations was assessed using a Bohlin CVO 50 Rheometer (Bohlin Instruments, UK) with parallel plate geometry (diameter 20 mm). Samples were carefully placed on the centre of the rheometer stage. The rotating element was then gently lowered to a distance of 1550 μm or 2050 μm, excess sample was trimmed off and the rotating element was lowered a further 50 μm to produce gap sizes of 1500 μm or 2000 μm, respectively. Samples were then left for 2 minutes before measurement to reach a temperature of 25° C. and to reduce any stress generated during sample loading. Gap size for fresh paste was carried out at a gap distance of 1500 μm, while the measurement for steamed and frozen products was performed at 2000 μm. Dynamic (oscillation) experiments were carried out to measure the response of the material to stress.

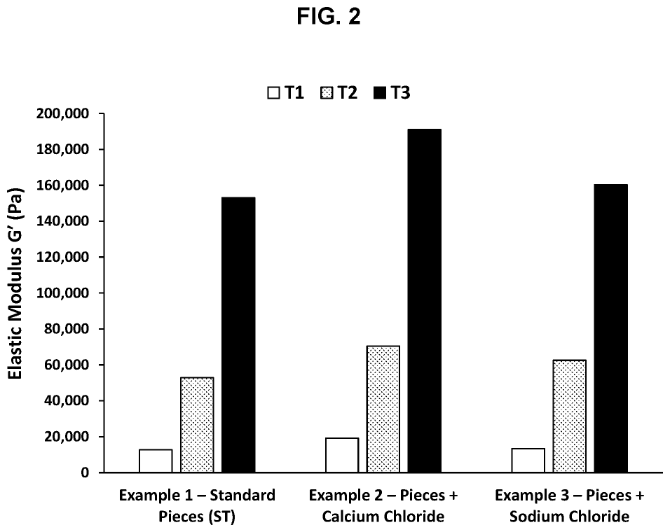

Results are provided in FIG. 2, which shows:
(i) That the Example 2 formulation, which includes calcium ions and mycoprotein alone, produces an increase in G′ (Pa) compared to mycoprotein, in the absence of calcium ions. This applies even for formulations stored under ambient conditions of Treatment 1 (T1). The effect on G′ is enhanced when treatments T2 and T3 are used.
(ii) The G′ (Pa) is increased if samples are steamed and/or frozen.
(iii) An increase in G′ (Pa) is also seen when sodium ions are included in formulations (compare Examples 1 and 3) although the effect is not as pronounced as when calcium ions are included as per Example 2.

While not being bound by any theory, in general terms, it is thought that cation binding to filamentous fungi is a physico-chemical interaction between the metal and the functional groups present on the fungi cell surface. For example, many fungi have high chitin content in their cell wall, and this polymer of N-acetyl glucosamine may interact with cations. Melanins are fungal pigments, located in and/or exterior to cell walls where they may appear as electron-dense deposits and granules. Fungal phenolic polymers and melanins contain phenolic units, peptides, carbohydrates, aliphatic hydrocarbons, and fatty acids and therefore possess many potential cation binding sites. Oxygen-containing groups in these substances, including carboxyl, phenolic and alcoholic hydroxyl, carbonyl and methoxy groups may be particularly important in cation binding.

There are several hypotheses that explain the effects observed with calcium ions. Fungal cell walls are predominantly net negative in surface charge due to the presence of relevant functional groups (e.g., uronic acids, which are in Fusarium walls, or phosphate, which is usually in the form of phosphodiester linkers to mannosyl carbohydrates that can be cleaved by mild acid treatment). The cations added to the system may effectively change the nature of the cell wall surface charge by reducing the net negative charge and thus reducing electrostatic repulsion between hyphae of the filamentous fungus. This in turn may promote ease of hyphal interaction (and a “tightening” of texture).

### Example 5

### General Process for Product Preparation

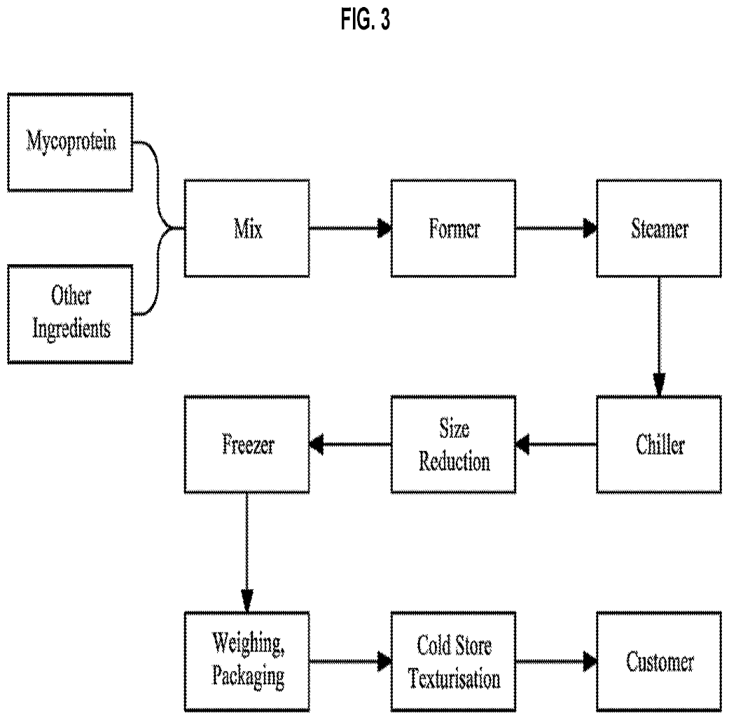

This is summarized in FIG. 3. Mycoprotein paste is mixed with other ingredients it is desired to incorporate to produce a substantially homogenous mass of a mycoprotein-containing foodstuff (e.g., meat-like pieces, mince, sausages, and roast meats). The homogenous mass is put through a former and then a steamer (e.g., over 95° C. for 35-45 minutes). The steamed product is then chilled (e.g., −5 to −10° C. for about 20 minutes), which improves the texture of the product by making it slightly firmer. There follows an optional size reduction process followed by a second texturization step involving freezing. Thereafter, products are weighed and packaged prior to the final texturization step at −18° C. in a cold store for at least 7 days. Thereafter product can be delivered to retail outlets for sale to customers.

### Example 6

### Preparation of Mycoprotein-Containing Pieces

Following the general procedure described in Example 5, the ingredients referred to in Table 1 were combined to produce the final product.

TABLE 1
Vegan Pieces Recipe

| | Wet | Wet Mass | Dry | Dry Mass |
|---|---|---|---|---|
| | Mass | Percentage | Mass | Percentage |
| Ingredient | (g/kg)* | (%) | (g/kg)** | (%) |
| Mycoprotein Paste | 884.20 | 88.4% | 221.00 | 70.7% |
| Water | 12.00 | 1.2% | <1.00^{†} | <0.3% |
| Flavour 1 | 10.00 | 1.0% | 9.50 | 3.0% |
| Potato Protein | 32.00 | 3.2% | 30.40 | 9.7% |
| Vital Wheat Gluten | 10.00 | 1.0% | 9.50 | 3.0% |
| Calcium Acetate | 4.00 | 0.4% | 3.80 | 1.2% |
| Calcium Chloride | 12.00 | 1.2% | 4.30 | 1.4% |
| Solution | | | | |
| Sodium Alginate | 0.80 | 0.1% | 0.76 | 0.2% |
| Flavour 2 | 3.00 | 0.3% | 2.85 | 0.9% |
| Carageenan | 4.00 | 0.4% | 3.80 | 1.2% |
| Wheat Fibre | 20.00 | 2.0% | 19.00 | 6.1% |
| Pea Fibre | 8.00 | 0.8% | 7.60 | 2.4% |
| Total | 1000.00 | 100.0% | 312.60 | 100.0% |

*Wet mass is the sum of the dry ingredient mass plus the total mass of water in the individual component.
**Dry mass is the dry ingredient mass in the absence of any water.
^{†}The water is not distilled/deionized and contains dissolved solids, thus upon evaporation the dissolved solids have some mass.

It should be appreciated that the steaming/chilling process affects the level of water in the final product which is generally in the range 70 to 77 wt % in total.

### Example 7

### Preparation of Mycoprotein-Containing Mince

Following the general procedure described in Example 5, the ingredients referred to in Table 2 were combined to produce the final product.

TABLE 2
Vegan Mince Recipe

| | Wet | Wet Mass | Dry | Dry Mass |
|---|---|---|---|---|
| | Mass | Percentage | Mass | Percentage |
| Ingredient | (g/kg) | (%) | (g/kg) | (%) |
| Mycoprotein Paste | 888.20 | 88.8% | 222.00 | 69.2% |
| Water | 0.00 | 0.0% | 0.00 | 0.0% |
| Potato Protein | 50.00 | 5.0% | 47.50 | 14.8% |
| Calcium acetate | 4.00 | 0.4% | 3.80 | 1.2% |
| Calcium Chloride Sodium | 12.00 | 1.2% | 4.20 | 1.3% |
| Sodium Alginate | 0.80 | 0.1% | 0.76 | 0.2% |
| Vital Wheat Gluten | 10.00 | 1.0% | 9.50 | 3.0% |
| Malt Extract | 6.00 | 0.6% | 5.70 | 1.8% |
| Caramelized Sugar | 9.00 | 0.9% | 8.55 | 2.7% |
| Wheat Fibre | 14.00 | 1.4% | 13.30 | 4.1% |
| Flavour | 2.00 | 0.2% | 1.90 | 0.6% |
| Carrageenan | 4.00 | 0.4% | 3.80 | 1.2% |
| Total | 1000.00 | 100.0% | 321.00 | 100.0% |

### Example 8

### Preparation of Mycoprotein-Containing Burger

Following the general procedure described in Example 5, the ingredients referred to in Table 3 were combined to produce the final product.

TABLE 3
Vegan Burger Recipe

| | Wet | Wet Mass | Dry | Dry Mass |
|---|---|---|---|---|
| | Mass | Percentage | Mass | Percentage |
| Ingredient | (g/kg) | (%) | (g/kg) | (%) |
| Mycoprotein Paste | 385.20 | 38.5% | 176.60 | 38.8% |
| Water | 171.10 | 17.1% | <1.00 | <0.2% |
| Onions | 100.00 | 10.0% | 15.00 | 3.3% |
| Meatless Mince | 83.30 | 8.3% | 16.70 | 3.7% |
| Malt Extract | 10.00 | 1.0% | 9.50 | 2.1% |
| Texturized Wheat Protein | 83.30 | 8.3% | 79.10 | 17.4% |
| Oil | 20.50 | 2.1% | 20.50 | 4.5% |
| Flavour 1 | 30.00 | 3.0% | 28.50 | 6.3% |
| Flaked Fat | 38.88 | 3.9% | 36.90 | 8.1% |
| Calcium Chloride Solution | 3.90 | 0.4% | 1.40 | 0.3% |
| Calcium Acetate | 3.90 | 0.4% | 3.70 | 0.8% |
| Flavour 2 | 3.00 | 0.3% | 2.90 | 0.6% |
| Wheat Fibre | 20.00 | 2.0% | 19.00 | 4.2% |
| Carrageenan | 4.00 | 0.4% | 3.80 | 0.8% |
| Sodium Alginate | 0.80 | 0.1% | 0.76 | 0.2% |
| Vital Wheat Gluten | 10.00 | 1.0% | 9.50 | 2.1% |
| Potato Protein | 32.00 | 3.2% | 30.40 | 6.7% |
| Total | 1000.00 | 100.0% | 455.00 | 100.0% |

### Example 9

### Preparation of Mycoprotein-Containing Sausage

Following the general procedure described in Example 5, the ingredients referred to in Table 4 were combined to produce the final product.

TABLE 4
Vegan Sausage Recipe

| | Wet | Wet Mass | Dry | Dry Mass |
|---|---|---|---|---|
| | Mass | Percentage | Mass | Percentage |
| Ingredient | (g/kg) | (%) | (g/kg) | (%) |
| Mycoprotein Paste | 431.60 | 43.2% | 107.90 | 26.6% |
| Water | 170.00 | 17.0% | <1.00 | <0.2% |
| Oil | 60.00 | 6.0% | 60.00 | 14.8% |
| Onions | 62.50 | 6.3% | 9.40 | 2.3% |
| Pea Fibre | 6.00 | 0.6% | 5.70 | 1.4% |
| Textured Wheat | 37.50 | 3.8% | 35.60 | 8.8% |
| Protein | | | | |
| Rusk | 68.80 | 6.9% | 65.30 | 16.1% |
| Meatless Mince | 43.80 | 4.4% | 8.80 | 2.2% |
| Tapioca Starch | 10.00 | 1.0% | 9.50 | 2.3% |
| Seasoning | 31.20 | 3.1% | 29.70 | 7.3% |
| Calcium Chloride | 4.40 | 0.4% | 1.40 | 0.3% |
| Solution | | | | |
| Calcium Acetate | 4.40 | 0.4% | 4.20 | 1.0% |
| Flavour | 3.00 | 0.3% | 2.80 | 0.7% |
| Wheat Fibre | 20.00 | 2.0% | 19.00 | 4.7% |
| Carrageenan | 4.00 | 0.4% | 3.80 | 0.9% |
| Sodium Alginate | 0.80 | 0.1% | 0.76 | 0.2% |
| Vital Wheat Gluten | 10.00 | 1.0% | 9.50 | 2.3% |
| Potato Protein | 32.00 | 3.2% | 30.40 | 7.5% |
| Total | 1000.00 | 100.0% | 405.00 | 100.0% |

In each of Examples 6-9, commercial products were produced which had excellent texture.

What is claimed:

1. A meat substitute edible formulation comprising:
at least 20 wt % on a dry mass basis of edible filamentous fungus particles comprising filaments of fungal mycelia;
at least 0.100 wt % and less than 1 wt % on a dry mass basis of calcium ions;
at least 50 wt % of water; and
acetate moieties;
wherein the formulation contains no animal derived components.

2. The formulation of claim 1, wherein the filaments of fungal mycelia have an aspect ratio of greater than 10 and a length of greater than 100 μm.

3. The formulation of claim 1, wherein the formulation comprises at least 2,000 mg of calcium ions per kg of filamentous fungus particles on a dry mass basis.

4. The formulation of claim 3, wherein the formulation comprises at least 5,000 mg of calcium ions per kg of filamentous fungus particles on a dry mass basis.

5. The formulation of claim 1, wherein the formulation comprises a ratio of the wt % on a dry mass basis of acetate ions divided by the wt % on a dry mass basis of filamentous fungus particles of at least 0.005.

6. The formulation of claim 1, wherein the formulation comprises at least 0.10 wt % on a dry mass basis and less than 1.5 wt % on a dry mass basis of acetate ions.

7. The formulation of claim 1, wherein the filamentous fungal particles have an RNA content of less than 1.9 wt % on a dry mass basis.

8. The formulation of claim 1, wherein the formulation comprises a ratio of the wt % on a dry mass basis of filamentous fungus particles divided by the wt % of water of at least 0.05 and less than 0.5.

9. The formulation of claim 1, wherein the formulation is provided in a package comprising at least 50 g of the formulation.

10. The formulation of claim 1, wherein at least 80 wt % of the filamentous fungal particles in the formulation comprise filaments of fungal mycelia.

11. The formulation of claim 1, wherein the filamentous fungal particles comprise fungi imperfecti.

12. The formulation of claim 1, wherein:
the filaments of fungal mycelia have an aspect ratio of greater than 10 and a length of greater than 100 μm;
the formulation comprises at least 2,000 mg of calcium ions per kg of filamentous fungus particles on a dry mass basis;
the formulation comprises a ratio of the wt % on a dry mass basis of acetate ions divided by the wt % on a dry mass basis of filamentous fungus particles of at least 0.005;
the formulation comprises at least 0.10 wt % on a dry mass basis and less than 1.5 wt % on a dry mass basis of acetate ions;
the filamentous fungal particles have an RNA content of less than 1.9 wt % on a dry mass basis; and
the formulation is provided in a package comprising at least 50 g of the formulation.

13. The formulation of claim 12, wherein:
at least 80 wt % of the filamentous fungal particles in the formulation comprise filaments of fungal mycelia;
the filamentous fungal particles comprise cells of a Fusarium species; and
the formulation is provided in a package comprising at least 100 g of the formulation.

14. A meat substitute edible formulation comprising:
at least 20 wt % on a dry mass basis of edible filamentous fungus particles;
calcium ions; and
acetate moieties;
wherein:
at least 80 wt % of the filamentous fungus particles comprise fungal mycelia;
the formulation comprises at least 2,000 mg of calcium ions per kg of filamentous fungus particles on a dry mass basis; and
a ratio of the wt % on a dry mass basis of acetate ions divided by the wt % on a dry mass basis of filamentous fungus particles is at least 0.005 and is less than 0.04.

15. The formulation of claim 14, wherein at least 80 wt % of the filamentous fungus particles comprise fungal mycelia, and the filamentous fungus particles are fungi imperfecti.

16. The formulation of claim 14, wherein the formulation is provided in a package comprising at least 100 g of the formulation.

17. The formulation of claim 14, wherein the filamentous fungus particles comprise cells of a Fusarium species, and the formulation is provided in a package comprising at least 50 g of the formulation.

18. The formulation of claim 14, wherein the formulation comprises at least 40 wt % on a dry mass basis of filamentous fungus particles.

19. The formulation of claim 14, wherein the formulation comprises at least 0.100 wt % and less than 1 wt % on a dry mass basis of calcium ions, and at least 50 wt % of water.

20. A meat substitute edible formulation comprising:
at least 20 wt % on a dry mass basis of edible filamentous fungus particles, wherein at least 99 wt % of the filamentous fungus particles comprise filaments of fungal mycelia having an aspect ratio of greater than 40 and a number average length of greater than 100 μm;
at least 6,000 mg of calcium ions per kg of filamentous fungus particles on a dry mass basis; and
at least 50 wt % of water;
wherein the formulation contains no animal derived components.
